(19) United States
(12) Patent Application Publication
(10) Pub. No.: US 2021/0043327 A1
(43) Pub. Date: Feb. 11, 2021

(54) COMPUTING DEVICE, PORTABLE DEVICE AND COMPUTER-IMPLEMENTED METHOD FOR PREDICTING MAJOR ADVERSE CARDIOVASCULAR EVENTS

(71) Applicant: TAIPEI MEDICAL UNIVERSITY, Taipei City (TW)

(72) Inventors: Yu-Chuan LI, TAIPEI CITY (TW); Chieh-Chen WU, TAIPEI CITY (TW)

(21) Appl. No.: 16/787,053

(22) Filed: Feb. 11, 2020

Related U.S. Application Data
(60) Provisional application No. 62/883,665, filed on Aug. 7, 2019.

(51) Int. Cl. G16H 50/50; G16H 50/30

(52) U.S. Cl. CPC G16H 50/50; G16H 50/30

(57) ABSTRACT

The present disclosure provides computing device, portable device and computed-implemented method for predicting and monitoring Major Adverse Cardiovascular Events (MACE). A MACE prediction model is generated according to a machine learning scheme with training data of a plurality of user data. A MACE occurrence level is determined according to selected variables associated with MACE.

### CROSS REFERENCE TO RELATED APPLICATIONS

This application claims priority of U.S. provisional application Ser. No. 62/883,665 filed on Aug. 7, 2019, which is incorporated by reference in its entirety.

### TECHNICAL FIELD

The present disclosure relates generally to a computing device, a portable device and a computer-implemented method for predicting condition of user, more particularly, to a computing device, a portable device and a computer-implemented method for predicting Major Adverse Cardiac Events (MACE) of user.

### BACKGROUND

Hospital or clinic may run medical check on people who suffer chest pain. Some people can be diagnosed as having cardiovascular disease. The patient(s) who have acute symptom may be hospitalized to receive treatment.

However, it would be challenging to determine whether an inpatient (who may have been treated) or a person (who may have cardiovascular disease but needs no immediate treatment) can be discharged. Even the patient is discharged, Major Adverse Cardiac Events (MACE) could still happen, which may jeopardize health or even life of the patient.

Although some rules have been developed or introduced to determine MACE that could occur on patients, however, there is still a huge room to improve accuracy of determination.

### SUMMARY

Some embodiments of the present disclosure provide a computing device for generating a Major Adverse Cardiac Events (MACE) prediction model. The computing device includes a processor and a storing unit. The storing unit stores a program that, when being executed, cause the processor to: retrieve a variable set, wherein the variable set includes a plurality of variables associated with MACE; determine a plurality of selected variables according to a feature selection model, wherein the selected variables include a corrected QT interval (QTc) variable; and generate the MACE prediction model according to a machine learning scheme with training data of a plurality of user data, wherein each user data includes a training input data and a training output data, the training input data corresponds to the plurality of selected variables and the training output data includes a MACE occurrence value.

Some embodiments of the present disclosure provide a computer-implemented method for generating a MACE prediction model. The computer-implemented method includes: inputting a variable set into a feature selection model, wherein the variable set includes a plurality of variables associated with MACE; determining a plurality of selected variables according to the feature selection model, wherein the selected variables include a QTc variable; generating the MACE prediction model according to a machine learning scheme with training data of a plurality of user data, wherein each user data includes a training input data and a training output data, the training input data corresponds to the plurality of selected variables and the training output data includes a MACE occurrence value.

Some embodiments of the present disclosure provide a portable device for predicting MACE. The portable device includes a sensor, processor and a storing unit. The sensor monitors cardio-data of a user. The storing unit stores a program that, when executed, causes the processor to: retrieve the cardio-data from the sensor; calculate a QTc according to the cardio-data; determine a MACE occurrence level according to the QTc.

Some embodiments of the present disclosure provide a computer-implemented method for predicting a MACE. The computer-implemented method includes: monitoring cardio-data of a user; calculating a QTc according to the cardio-data; determining a MACE occurrence level according to the QTc.

The foregoing has outlined rather broadly the features and technical advantages of the present disclosure in order that the detailed description of the disclosure that follows may be better understood. Additional features and advantages of the disclosure will be described hereinafter, and form the subject of the claims of the disclosure. It should be appreciated by those skilled in the art that the conception and specific embodiment disclosed may be readily utilized as a basis for modifying or designing other structures or processes for carrying out the same purposes of the present disclosure. It should also be realized by those skilled in the art that such equivalent constructions do not depart from the spirit and scope of the disclosure as set forth in the appended claims.

### BRIEF DESCRIPTION OF THE DRAWINGS

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion.

A more complete understanding of the present disclosure may be derived by referring to the detailed description and claims when considered in connection with the Figures, where like reference numbers refer to similar elements throughout the Figures.

FIG. 1A is a block diagram of a computing device according to some embodiments of the present disclosure.

FIG. 1B is a schematic view of selecting variables from a variable set according to some embodiments of the present disclosure.

FIG. 2A is a block diagram of a portable device according to some embodiments of the present disclosure.

FIG. 2B is a block diagram of a portable device according to some embodiments of the present disclosure.

FIG. 3 is a flowchart diagram of a computer-implemented method according to some embodiments of the present disclosure.

FIG. 4 is a flowchart diagram of a computer-implemented method according to some embodiments of the present disclosure.

FIGS. 5A and 5B are flowchart diagrams of a computer-implemented method according to some embodiments of the present disclosure.

FIGS. 6A and 6B are flowchart diagrams of a computer-implemented method according to some embodiments of the present disclosure.

### DETAILED DESCRIPTION

Embodiments, or examples, of the disclosure illustrated in the drawings are now described using specific language. It shall be understood that no limitation of the scope of the disclosure is hereby intended. Any alteration or modification of the described embodiments, and any further applications of principles described in this document, are to be considered as normally occurring to one of ordinary skill in the art to which the disclosure relates. Reference numerals may be repeated throughout the embodiments, but this does not necessarily mean that feature(s) of one embodiment apply to another embodiment, even if they share the same reference numeral.

It shall be understood that, although the terms first, second, third, etc. may be used herein to describe various elements, components, regions, layers or sections, these elements, components, regions, layers or sections are not limited by these terms. Rather, these terms are merely used to distinguish one element, component, region, layer or section from another element, component, region, layer or section. Thus, a first element, component, region, layer or section discussed below could be termed a second element, component, region, layer or section without departing from the teachings of the present inventive concept.

The terminology used herein is for the purpose of describing particular example embodiments only and is not intended to be limited to the present inventive concept. As used herein, the singular forms “a,” “an” and “the” are intended to include the plural forms as well, unless the context clearly indicates otherwise. It shall be further understood that the terms “comprises” and “comprising,” when used in this specification, point out the presence of stated features, integers, steps, operations, elements, or components, but do not preclude the presence or addition of one or more other features, integers, steps, operations, elements, components, or groups thereof.

Major Adverse Cardiac Events (MACE) is a composite of all-cause mortality associated with cardiovascular-related illnesses. Some Standard Operating Procedure (SOPs) for determining MACE are applied to the patients, who mainly complain about chest pain, in the emergency department of the hospital.

However, these SOPs can be relatively imprecise in determination of MACE. Therefore, new method and operating device for relatively, precisely determining MACE are required.

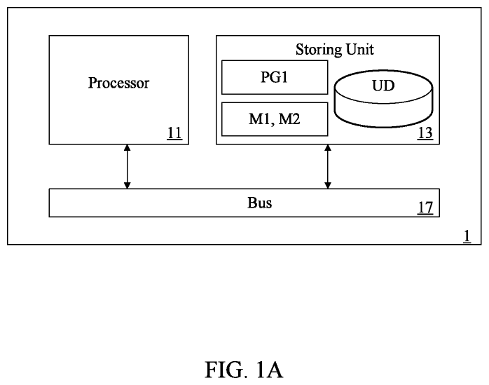

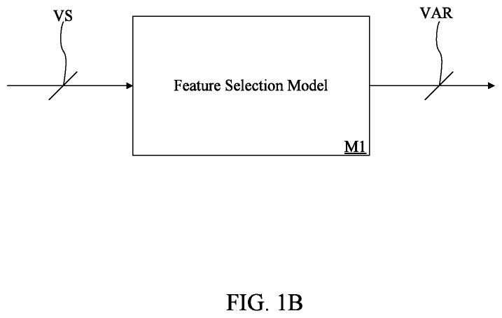

FIG. 1A illustrates a block diagram of a computing device 1 according to some embodiments of the present disclosure. The computing device 1 includes a processor 11 and a storing unit 13. The processor 11 and the storing unit 13 are electrically coupled through a communication bus 17.

The communication bus 17 may allow the processor 11 to execute a program PG1 stored in the storing unit 13. When executed, the program PG1 may generate one or more interrupts (e.g., software-interrupt) to cause the processor 11 to perform functions of the program PG1 for generating MACE prediction model. The functions of the program PG1 will be further described hereinafter.

In some embodiments, before generating a MACE prediction model, significant biological variables related with MACE should be selected for generating the MACE prediction model. In particular, although many biological variables (e.g., age, gender, diabetes, sodium, potassium, etc.) may be associated with MACE, some of these biological variables are less significant to MACE. Therefore, critical biological variables should be selected first for predicting MACE.

In detail, a variable set VS which includes a plurality of variables associated with MACE is provided. To further reduce the less significant variables, technique of feature selection is introduced. More specifically, a feature selection model M1 stored in the storing unit 13 is utilized to select some significant variables from the variable set VS.

Accordingly, as shown in FIG. 1B, the program PG1 causes the processor 11 to input the variables set VS into the feature selection model M1. Next, the program PG1 causes the processor 11 to determine a plurality of selected variables VAR according to the output of the feature selection model M1. In some embodiments, the selected variables VAR include a corrected QT interval (QTc) variable. In other words, QTc variable is a significant variable for predicting MACE.

In some embodiments, the feature selection model M1 selects the selected variables VAR according to mutual information of the variable set VS. In particular, as for each variable “X” of the variable set VS, the mutual information includes dependence between the variable “X” and other variables, and the dependence can be quantified as dependence values.

More specifically, the dependence between the variable “X” and the variable “Y” can be quantified as a dependence value “D_{xy}” from “0” to “1”. If the dependence value “D_{xy}” is equal to “0”, it means that the variable “X” is independent of the variable “Y”. On the other hand, the higher the dependence value “D_{xy}” is, the dependence between the variable “X” and the variable “Y” is higher.

In some embodiments, the feature selection model M1 selects the selected variables VAR according to a recursive feature elimination model. In particular, the recursive feature elimination model utilizes importance scores, which correspond to the variables of the variable set VS, based on a machine learning model and sorts the importance scores from highest to lowest.

Next, the least important feature is pruned from the variable set VS and newly scores are estimated for the variables of the variable set VS. This process is repeated until the number of remaining variables of the variable set VS is equal to a required number.

The recursive feature elimination model may be Support Vector Classification (SVC) model, Logistic Regression (LR) model, Ridge Classification (RC) model or Random Forest (RF) model. However, it is not intended to limit the implementation of the feature selection model of the present disclosure.

It should be noted that the data of the mutual information or the importance scores of the recursive feature elimination model may be collected from some clinical data of patients. The people skilled in the art should easily understand how to use these data according to the above disclosure.

After the selected variables VAR are determined, a MACE prediction model M2 for predicting occurrence of MACE can be trained according to some training data corresponding to the selected variables VAR.

In particular, because the MACE prediction model M2 should be used to receive biological data of a user and output an occurrence of MACE for the user, a plurality of biological data and corresponding occurrences of MACE should be used as the training data for training the MACE prediction model M2.

Further, since the selected variables VAR are determined as significant variables for MACE, the biological data used for training the MACE prediction model M2 should correspond to the selected variables VAR.

More specifically, the MACE prediction model M2 should be trained according to a machine learning scheme with training data that includes: (1) biological data corresponding to the selected variables VAR; and (2) MACE occurrence values. In some embodiments, the MACE occurrence value may include a Boolean which is used to indicate positive of MACE happening or negative of MACE happening.

Accordingly, in some embodiments, a plurality of user data UD stored in the storing unit 13 can be used for training the MACE prediction model M2. Each user data UD includes: (1) biological data, which corresponds to the selected variables VAR, of a user; and (2) a MACE occurrence value of the same user.

Further, the biological data corresponding to the selected variables VAR are used as training input data for training the MACE prediction model M2. The MACE occurrence values are used as training output data for training the MACE prediction model M2.

More particularly, the program PG1 causes to processor 11 to generate (i.e., to train) the MACE prediction model M2 according to the user data UD. The biological data, which correspond to the selected variables VAR, of the user data UD are used as training input data during the training stage. The MACE occurrence values of the user data UD are used as training output data during the training stage. After the processor 11 generates the MACE prediction model M2, the storing unit 13 stores the MACE prediction model M2 for later use.

It should be note that, in some embodiments, an Artificial Neural Networks (ANN) algorithm that is capable of building a model for predicting a result based on the user data is introduced for generating the MACE prediction model M2.

In particular, in the implementation (e.g., program code) of the ANN algorithm for training the MACE prediction model M2, there is a training function (e.g., a function of the program code) for training the MACE prediction model M2. During the training of the MACE prediction model M2, the training function includes a section (e.g., part of the function) for receiving the user data UD.

Further, biological data, which correspond to the selected variables VAR, of the user data UD are used as training input data. MACE occurrence values of the user data UD are used as training output data. Next, the MACE prediction model M2 can be trained after the training function is executed with a main function (e.g., a main part of the program code) of the implementation of the ANN algorithm.

After generating the MACE prediction model M2 with the training data (i.e., the user data UD) according to the ANN algorithm, the MACE prediction model M2 can be used for predicting an occurrence of MACE for a user.

For example, when it is needed to predict whether MACE happens to a user in the near future, biological data, which correspond to the selected variables VAR including QTc variable, of the user are inputted into the MACE prediction model M2. The MACE prediction model M2 then outputs a MACE occurrence value for the user.

If the MACE occurrence value is negative, it means that MACE may not happen to the user in the near future. On the other hand, if the MACE occurrence value is positive, it means that MACE may happen to the user in the near future.

It should be noted that, in some embodiments, a time factor may be introduced for generating the MACE prediction model M2. In detail, in these embodiments, the MACE occurrence value further indicates whether MACE happens to the user within a period. Accordingly, after the MACE prediction model M2 is generated, the MACE prediction model M2 can be used for predicting whether MACE happens to the user within the period.

For ease of understanding the mentioned technologies of the present disclosure, an example will be demonstrated hereinafter. In some embodiments, before generating MACE prediction model, critical biological variables should be selected first for predicting MACE.

In detail, the variable set VS includes 37 variables which are experimentally associated with MACE. To further reduce the less significant variables, technique of feature selection is applied. Accordingly, the program PG1 causes the processor 11 to input the variables set VS into the feature selection model M1. Next, the program PG1 causes the processor 11 to determine the selected variables VAR according to the output of the feature selection model M1.

In some embodiments, the selected variables VAR include the QTc variable, an age variable and a Coronary Artery Disease (CAD) risk factor variable. The CAD risk factor may include a number of CAD. It should be noted that these selected variables VAR are non-invasive type which means that these selected variables VAR can be obtained without invasive treatment.

After the selected variables VAR are determined, the MACE prediction model M2 can be trained according to some training data corresponding to the selected variables VAR. In particular, the program PG1 causes the processor 11 to generate the MACE prediction model M2 according to the machine learning scheme with training data of the user data UD. The user data UD correspond to the selected variables VAR which include the QTc variable, the age variable and the CAD risk factor variable.

In detail, the MACE prediction model M2 should be trained according to the machine learning scheme with the user data UD. Each user data UD includes: (1) biological data, which corresponds to the QTc variable, the age variable and the CAD risk factor variable, of a user; and (2) a MACE occurrence value of the same user. The MACE occurrence value indicates whether MACE happens to the user within three months.

Further, the biological data corresponding to the QTc variable, the age variable and the CAD risk factor variable are used as training input data for training the MACE prediction model M2. The MACE occurrence values are used as training output data for training the MACE prediction model M2.

After generating (i.e., training) the MACE prediction model M2 with the training data (i.e., the user data UD), the MACE prediction model M2 can be used for predicting an occurrence of MACE for a user.

For example, when it is needed to predict whether MACE may happen to a user within three months, biological data, which correspond to the QTc variable, the age variable and the CAD risk factor variable, of the user are inputted into the MACE prediction model M2. In other words, biological data which includes QTc data, age data and CAD risk factor of the user are inputted into the MACE prediction model M2. The MACE prediction model M2 then outputs a MACE occurrence value for the user.

If the MACE occurrence value is negative, it means that MACE may not happen to the user within three month. On the other hand, if the MACE occurrence value is positive, it means that MACE may happen to the user within three month.

In some embodiments, the selected variables VAR include the QTc variable, the age variable, the CAD risk factor variable, a creatinine variable and a troponin I variable. It should be noted that the creatinine variable and the troponin I variable are invasive type which means that these variables should be obtained with invasive treatment (e.g., blood test).

After the selected variables VAR are determined, the MACE prediction model M2 can be trained according to some training data corresponding to the selected variables VAR. In particular, the program PG1 causes the processor 11 to generate the MACE prediction model M2 according to the machine learning scheme with training data of the user data UD. The user data UD correspond to the selected variables VAR which include the QTc variable, the age variable, the CAD risk factor variable, the creatinine variable and the troponin I variable.

In detail, the MACE prediction model M2 should be trained according to the machine learning scheme with the user data UD. Each user data UD includes: (1) biological data, which corresponds to the QTc variable, the age variable, the CAD risk factor variable, the creatinine variable and the troponin I variable, of a user; and (2) a MACE occurrence value of the same user. The MACE occurrence value indicates whether MACE happens to the user within three months.

Further, the biological data corresponding to the QTc variable, the age variable, the CAD risk factor variable, the creatinine variable and the troponin I variable are used as training input data for training the MACE prediction model M2. The MACE occurrence values are used as training output data for training the MACE prediction model M2.

After generating (i.e., training) the MACE prediction model M2 with the training data (i.e., the user data UD), the MACE prediction model M2 can be used for predicting an occurrence of MACE for a user.

For example, when it is needed to predict whether MACE may happen to a user within three months, biological data, which correspond to the QTc variable, the age variable, the CAD risk factor variable, the creatinine variable and the troponin I variable, of the user are inputted into the MACE prediction model M2. In other words, biological data which includes QTc data, age data, CAD risk factor, creatinine data and troponin I data of the user are inputted into the MACE prediction model M2. The MACE prediction model M2 then outputs a MACE occurrence value for the user.

If the MACE occurrence value is negative, it means that MACE may not happen to the user within three month. On the other hand, if the MACE occurrence value is positive, it means that MACE may happen to the user within three month.

In some embodiments, a person who is diagnosed or determined as having symptom that may have relatively low risk of MACE at one moment, but a long-term monitoring would still be required in case MACE could happen in the future. Accordingly, new method and portable device should be developed for monitoring this patient and predicting MACE during a time period.

In particular, since the selected variables VAR which include QTc variable are determined as significant variables for predicting MACE and are verified as significant variables according to the machine learning model, data corresponding the selected variables VAR may be further classified in different levels for assisting in predicting MACE.

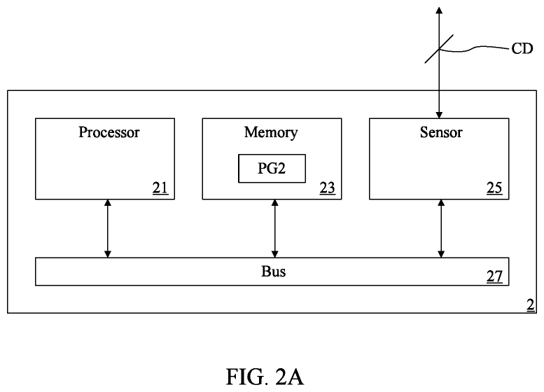

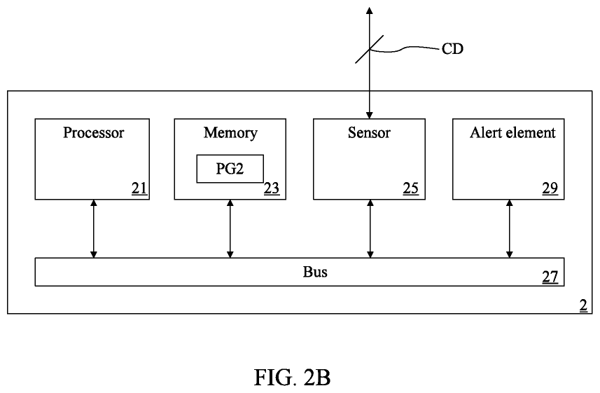

FIG. 2A illustrates a block diagram of a portable device 2 according to some embodiments of the present disclosure. The portable device 2 includes a processor 21, a memory 23 and a sensor 25. The processor 21, the memory 23 and the sensor 25 are electrically coupled through a communication bus 27.

The communication bus 27 may allow the processor 21 to execute a program PG2 stored in the memory 23. When executed, the program PG2 may generate one or more interrupts (e.g., software-interrupt) to cause the processor 21 to perform functions of the program PG2 for monitoring user for predicting possible happening of MACE. The functions of the program PG2 will be further described hereinafter.

In detail, the sensor 25 is used for sensing cardio-data CD of a user. The program PG2 causes the processor 21 to retrieve the cardio-data CD from the sensor 25. In these embodiments, the program PG2 causes the processor 21 to calculate a QTc according to the cardio-data CD because QTc is a significant variable for predicting MACE.

Next, the program PG2 causes the processor 21 to determine a MACE occurrence level according to the QTc. Accordingly, the user may understand a possibility of MACE occurrence based on the MACE occurrence level.

For ease of understanding the present disclosure, an example will be demonstrated hereinafter. In some embodiments, a score table (not shown) stored in the storing unit 23 is utilized for assisting the determination of the MACE occurrence level. In particular, according to the selected variables VAR which includes age variable, CAD risk factor variable and QTc variable, the score table includes an age score sub-table, a CAD risk factor score sub-table and a QTc score sub-table.

The age score sub-table indicates that: (1) age between “A1” to “A2” corresponds to score “0”; (2) age between “A2” to “A3” corresponds to score “1”; and (3) age greater than “A3” corresponds to score “2”.

The CAD risk factor score sub-table indicates that: (1) number of CAD risk factor “B1” corresponds to score “0”; (2) number of CAD risk factor “B2” corresponds to score “1”; and (3) number of CAD risk factor greater than “B3” corresponds to score “2”.

The QTc score sub-table indicates that: (1) QTc less than “C1” corresponds to score “0”; (2) QTc between “C1” to “C2” corresponds to score “1”; and (3) QTc greater than “C2” corresponds to score “2”.

Accordingly, for a user who uses the portable device 2, the user first inputs age and CAD risk factor into the portable device 2. The sensor 25 is used for sensing cardio-data CD of the user. Next, the program PG2 causes the processor 21 to retrieve the cardio-data CD from the sensor 25 and to calculate a QTc according to the cardio-data CD.

Then, the program PG2 causes the processor 21 to calculate a total score based on the score table. For instance, as for the user, when the age is between “A1” and “A2”, the number of the CAD risk factor is “B1” and the QTc is less than “C1”, the total score is 0+0+0=0. The MACE occurrence level may be determined as “Low Risk”.

For instance, as for the user, when the age is between “A2” and “A3”, the number of the CAD risk factor is “B2” and the QTc is between “C1” and “C2”, the total score is 1+1+1=3. The MACE occurrence level may be determined as “Medium Risk”.

For instance, as for the user, when the age is greater than “A3”, the number of the CAD risk factor is greater than “B3” and the QTc is greater than “C3”, the total score is 2+2+2=6. The MACE occurrence level may be determined as “High Risk”.

For some examples, the MACE occurrence level may be classified: (1) as “Low Risk” when the total score is between “0” to “1”; (2) as “Medium Risk” when the total score is between “2” to “3”; and (3) as “High Risk” when the total score is between “4” to “6”.

It should be noted that, because the age and the CAD risk factor are less variable, the change of the total score may mainly depend on QTc.

In some embodiments, another score table (not shown) is utilized for assisting the determination of the MACE occurrence level. In particular, according to the selected variables VAR which includes age variable, CAD risk factor variable, QTc variable, creatinine variable and troponin I variable, the score table includes an age score sub-table, a CAD risk factor score sub-table, a QTc score sub-table, a creatinine score sub-table and a troponin I score sub-table.

The age score sub-table indicates that: (1) age between “a1” to “a2” corresponds to score “0”; and (2) age greater than “a2” corresponds to score “1”.

The CAD risk factor score sub-table indicates that: (1) number of CAD risk factor “b1” corresponds to score “0”; (2) number of CAD risk factor “b2” corresponds to score “1”; and (3) number of CAD risk factor greater than “b3” corresponds to score “2”.

The QTc score sub-table indicates that: (1) QTc less than “c1” corresponds to score “0”; and (2) QTc greater than “c1” corresponds to score “2”.

The creatinine score sub-table indicates that: (1) creatinine between “d1” to “d2” corresponds to score “0”; and (2) creatinine greater than “d2” corresponds to score “2”.

The troponin I score sub-table indicates that: (1) troponin I less than “e1” corresponds to score “0”; and (2) troponin I greater than “e1” corresponds to score “3”.

Accordingly, for a user who uses the portable device 2, the user inputs age, CAD risk factor, creatinine and troponin I into the portable device 2. The sensor 25 is used for sensing cardio-data CD of the user. Next, the program PG2 causes the processor 21 to retrieve the cardio-data CD from the sensor 25 and to calculate a QTc according to the cardio-data CD.

Then, the program PG2 causes the processor 21 to calculate a total score based on the score table. For instance, as for the user, when the age is between “a1” and “a2”, the number of the CAD risk factor is “b1”, the QTc is less than “c1”, the creatinine is between “d1” and “d2” and the troponin I is less than “e1”, the total score is 0+0+0+0+0=0. The MACE occurrence level may be determined as “Low Risk”.

For instance, as for the user, when the age is between “a1” and “a2”, the number of the CAD risk factor is “b3”, the QTc is greater than “c1”, the creatinine is between “d1” and “d2” and the troponin I is less than “e1”, the total score is 0+2+2+0+0=4. The MACE occurrence level may be determined as “Medium Risk”.

For instance, as for the user, when the age is greater than “a2”, the number of the CAD risk factor is “b3”, the QTc is greater than “c1”, the creatinine is greater than “d2” and the troponin I is less than “e1”, the total score is 1+2+2+2+0=7. The MACE occurrence level may be determined as “High Risk”.

For some examples, the MACE occurrence level may be classified: (1) as “Low Risk” when the total score is between “0” to “3”; (2) as “Medium Risk” when the total score is between “4” to “6”; and (3) as “High Risk” when the total score is between “7” to “10”.

It should be noted that, because the age and the CAD risk factor are less variable, the change of the total score may mainly depend on QTc, creatinine and troponin I. The creatinine and the troponin I are obtained by invasive treatment.

In some embodiments, the portable device 2 may alter user for MACE occurrence level. FIG. 2B illustrates a block diagram of the portable device 2 according to some embodiments of the present disclosure. The portable device 2 further includes an alert element 29. The processor 21, the memory 23, the sensor 25 and the alert element 29 are electrically coupled through a communication bus 27

In detail, the program PG2 causes the processor 21 to trigger the alert element 29 according to the MACE occurrence level. For example, when the MACE occurrence level is determined as “High Risk” by the processor 21, the program PG2 then causes the processor 21 to trigger the alert element 29. Therefore, the user can be alerted a high possibility of MACE occurrence.

In some embodiments, the alert element 29 may include a display for display the MACE occurrence level. In some embodiments, the alert element 29 may include a speaker for making sound notification of the MACE occurrence level. However, it is not intended to limit the implementation of the alert element of the present disclosure.

In some embodiments, the portable device 2 may include a wearable device. However, it is not intended to limit the hardware implementation embodiments of the present disclosure.

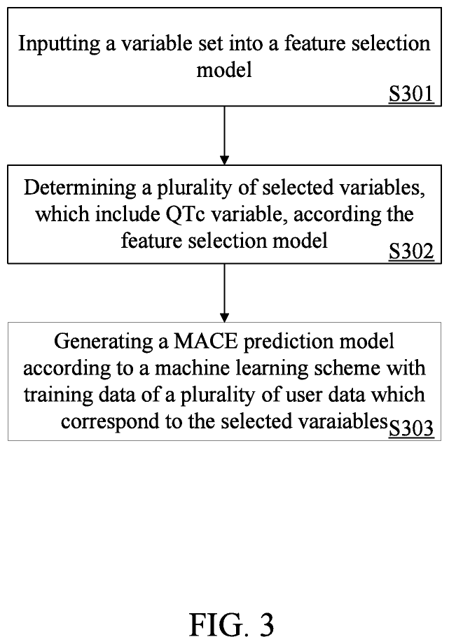

Some embodiments of the present disclosure include a computer-implemented method for generating a MACE prediction model, and a flowchart diagram thereof is shown in FIG. 3. The computer-implemented method of some embodiments is for use in a computing device (e.g., the computing device of the aforesaid embodiments). Detailed steps of the computer-implemented method are described below.

Step S301 is executed, by a processor of the computing device, to input a variable set into a feature selection model. The variable set includes a plurality of variables associated with MACE. Step S302 is executed, by the processor, to determine a plurality of selected variables according to the feature selection model. The selected variables include a QTc variable.

Step S303 is executed, by the processor, to generate the MACE prediction model according to a machine learning scheme with training data of a plurality of user data. Each user data includes a training input data and a training output data. The training input data corresponds to the plurality of selected variables and the training output data includes a MACE occurrence value. In some embodiments, the training output data includes the MACE occurrence value within a period.

In some embodiments, in step S301, the variable set may be inputted into the feature selection model with mutual information of the variable set. In particular, the mutual information corresponds to dependencies between the variables of the variable set.

In some embodiments, in step S301, the variable set may be inputted into the feature selection model with importance scores of the variables and the feature selection model may include a recursive feature elimination model for selecting the selected variables by the importance scores of the variables.

In some embodiments, the selected variables further include an age variable and a CAD risk factor variable. In some embodiments, the selected variables further include a creatinine variable and a troponin variable.

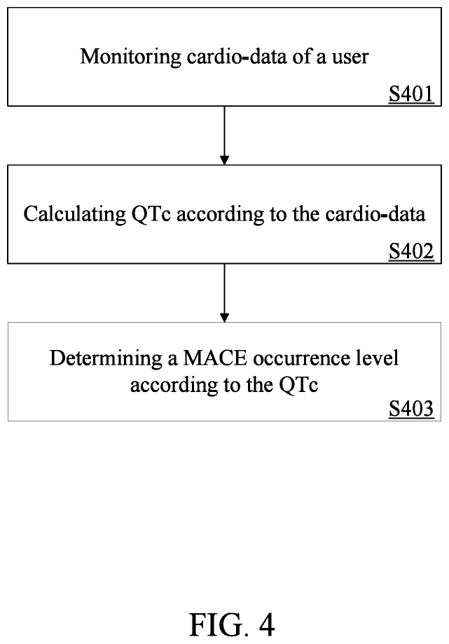

Some embodiments of the present disclosure include a computer-implemented method for predicting MACE, and a flowchart diagram thereof is shown in FIG. 4. The computer-implemented method of some embodiments is for use in a portable device (e.g., the portable device of the aforesaid embodiments). Detailed steps of the computer-implemented method are described below.

Step S401 is executed, by a sensor of the portable device, to monitor cardio-data of a user. Step S402 is executed, by a processor of the portable device, to calculate a QTc according to the cardio-data. Step S403 is executed, by the processor, to determine a MACE occurrence level according to the QTc.

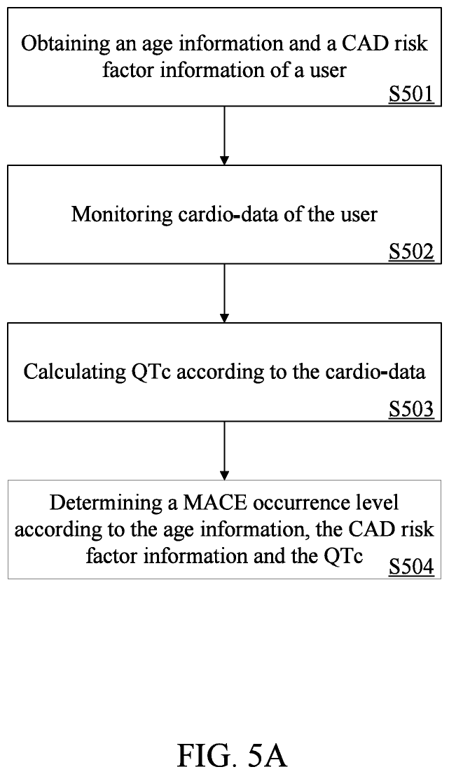

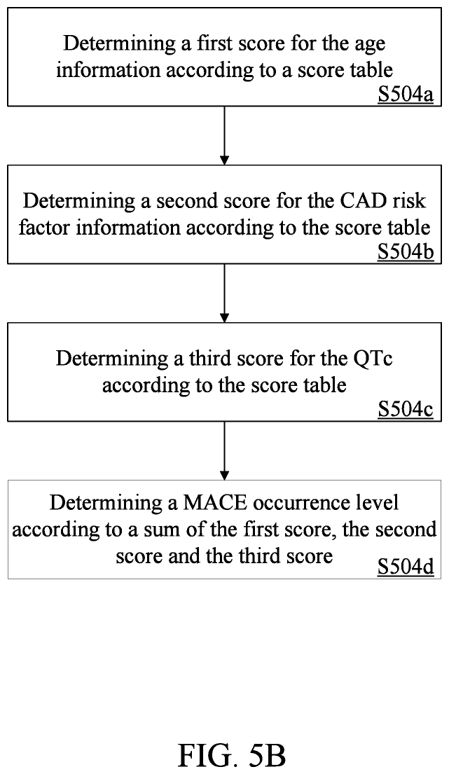

Some embodiments of the present disclosure include a computer-implemented method for predicting MACE, and flowchart diagrams thereof are shown in FIGS. 5A and 5B. The computer-implemented method of some embodiments is for use in a portable device (e.g., the portable device of the aforesaid embodiments). Detailed steps of the computer-implemented method are described below.

Step S501 is executed, by a processor of the portable device, to obtain an age information and a CAD risk factor information of the user. In some embodiments, the age information and the CAD risk factor information can be inputted into the portable device 2 by the user.

Step S502 is executed, by a sensor of the portable device, to monitor cardio-data of a user. Step S503 is executed, by the processor, to calculate a corrected QTc according to the cardio-data. Step S504 is executed, by the processor, to determine a MACE occurrence level according to the QTc, the age information and the CAD risk factor information.

In some embodiments, step S504 may further include the following steps. Step S504a is executed, by the processor, to determine a first score for the age information according to a score table stored in a storing unit of the portable device.

Step S504b is executed, by the processor, to determine a second score for the CAD risk factor information according to the score table. Step S504c is executed, by the processor, to determine a third score for the QTc according to the score table. Step S504d is executed, by the processor, to determine the MACE occurrence level according to a sum of the first score, the second score and the third score.

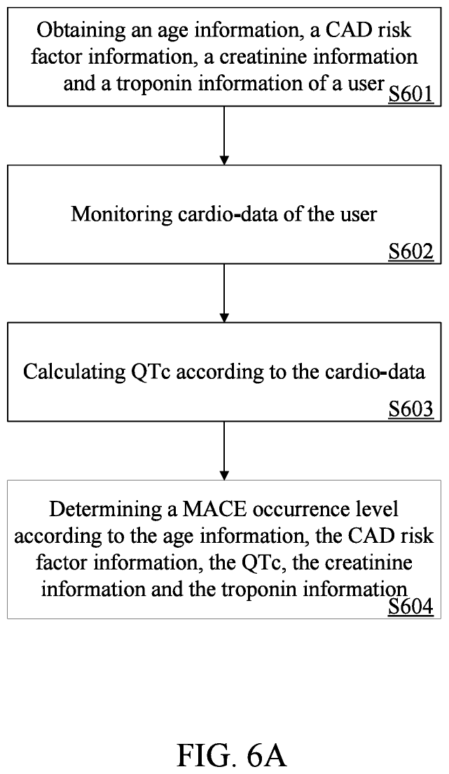

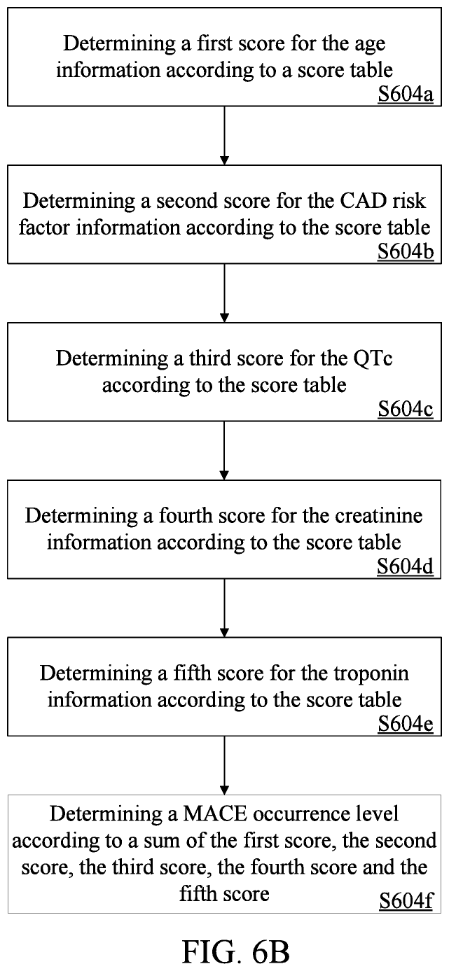

Some embodiments of the present disclosure include a computer-implemented method for predicting MACE, and flowchart diagrams thereof are shown in FIGS. 6A and 6B. The computer-implemented method of some embodiments is for use in a portable device (e.g., the portable device of the aforesaid embodiments). Detailed steps of the computer-implemented method are described below.

Step S601 is executed, by a processor of the portable device, to obtain an age information, a CAD risk factor information, a creatinine information and a troponin information of a user. In some embodiments, the age information, the CAD risk factor information, the creatinine information and the troponin information can be inputted into the portable device 2 by the user.

Step S602 is executed, by a sensor of the portable device, to monitor cardio-data of the user. Step S603 is executed, by the processor, to calculate a corrected QTc according to the cardio-data. Step S604 is executed, by the processor, to determine a MACE occurrence level according to the QTc, the age information and the CAD risk factor information.

In some embodiments, step S604 may further include the following steps. Step S604a is executed, by the processor, to determine a first score for the age information according to a score table stored in a storing unit of the portable device.

Step S604b is executed, by the processor, to determine a second score for the CAD risk factor information according to the score table. Step S604c is executed, by the processor, to determine a third score for the QTc according to the score table. Step S604d is executed, by the processor, to determine a fourth score for the creatinine information according to the score table.

Step S604e is executed, by the processor, to determine a fifth score for the troponin information according to the score table. Step S604f is executed, by the processor, to determine the MACE occurrence level according to a sum of the first score, the second score and, the third score, the fourth score and the fifth score.

It shall be particularly appreciated that the processors mentioned in the above embodiments may be a central processing unit (CPU), other hardware circuit elements capable of executing relevant instructions, or combination of computing circuits that are well-known by those skilled in the art based on the above disclosures.

Moreover, the storing units mentioned in the above embodiments may include memories, such as ROM, RAM, etc., or storing device, such as flash memory, HDD, SSD, etc., for storing data. Further, the communication buses mentioned in the above embodiments may include a communication interface for transferring data between the elements, such as the processor, the storing unit, the sensor and the alert element, and may include electrical bus interface, optical bus interface or even wireless bus interface. However, such description is not intended to limit the hardware implementation embodiments of the present disclosure.

Although the present disclosure and its advantages have been described in detail, it should be understood that various changes, substitutions and alterations can be made herein without departing from the spirit and scope of the disclosure as defined by the appended claims. For example, many of the processes discussed above can be implemented in different methodologies and replaced by other processes, or a combination thereof.

Moreover, the scope of the present application is not intended to be limited to the particular embodiments of the process, machine, manufacture, composition of matter, means, methods and steps described in the specification. As one of ordinary skill in the art will readily appreciate from the disclosure of the present disclosure, processes, machines, manufacture, compositions of matter, means, methods, or steps, presently existing or later to be developed, that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A computing device for generating a Major Adverse Cardiac Events (MACE) prediction model, comprising:
a processor; and
a storing unit including a program that, when being executed, causes the processor to:
retrieve a variable set, wherein the variable set includes a plurality of variables associated with MACE;
determine a plurality of selected variables according to a feature selection model, wherein the selected variables include a corrected QT interval (QTc) variable; and
generate the MACE prediction model according to a machine learning scheme with training data of a plurality of user data, wherein each user data includes a training input data and a training output data, the training input data corresponds to the plurality of selected variables and the training output data includes a MACE occurrence value.

2. The computing device of claim 1, wherein the processor further retrieves the variable set with mutual information, and the mutual information corresponds to dependencies between the variables.

3. The computing device of claim 1, wherein the feature selection model includes a recursive feature elimination model.

4. The computing device of claim 1, wherein the MACE occurrence value indicates whether MACE happens within a period.

5. The computing device of claim 1, wherein the selected variables further include an age variable and a Coronary Artery Disease (CAD) risk factor variable.

6. The computing device of claim 5, wherein the selected variables further include a creatinine variable and a troponin variable.

7. A computer-implemented method for generating a Major Adverse Cardiac Events (MACE) prediction model, comprising:
receiving a variable set, wherein the variable set includes a plurality of variables associated with MACE;
determining a plurality of selected variables according to a feature selection model, wherein the selected variables include a corrected QT interval (QTc) variable;
generating the MACE prediction model according to a machine learning scheme with training data of a plurality of user data, wherein each user data includes a training input data and a training output data, the training input data corresponds to the plurality of selected variables and the training output data includes a MACE occurrence value.

8. The computer-implemented method of claim 7, wherein inputting the variable set further comprises:
inputting the variable set with mutual information into the feature selection model, wherein the mutual information corresponds to dependencies between the variables.

9. The computer-implemented method of claim 7, wherein the feature selection model includes a recursive feature elimination model.

10. The computer-implemented method of claim 7, wherein the MACE occurrence value indicates whether MACE happens within a period.

11. The computer-implemented method of claim 7, wherein the selected variables further include an age variable and a Coronary Artery Disease (CAD) risk factor variable.

12. The computer-implemented method of claim 11, wherein the selected variables further include a creatinine variable and a troponin variable.

13. A portable device for predicting Major Adverse Cardiac Events (MACE), comprising:
a sensor, for monitoring cardio-data of a user;
a processor; and
a storing unit including a program that, when being executed, causes the processor to:
retrieve the cardio-data from the sensor;
calculate a corrected QT interval (QTc) according to the cardio-data;
determine a MACE occurrence level according to the QTc.

14. The portable device of claim 13, wherein the processor further determines the MACE occurrence level according to the QTc, an age information and a Coronary Artery Disease (CAD) risk factor information.

15. The portable device of claim 14, wherein the storing unit further stores a score table, and the program, when being executed, further causes the processor to:
determine a first score for the age information according to the score table;
determine a second score for the CAD risk factor information according to the score table;
determine a third score for the QTc according to the score table;
determine the MACE occurrence level according to a sum of the first score, the second score and the third score.

16. The portable device of claim 13, further comprising an alert element, wherein the program, when being executed, further causes the processor to trigger the alert element according to the MACE occurrence level.

17. A computer-implemented method for predicting a Major Adverse Cardiac Events (MACE), comprising:
monitoring cardio-data of a user;
calculating a corrected QT interval (QTc) according to the cardio-data;
determining a MACE occurrence level according to the QTc.

18. The computer-implemented method of claim 17, further comprising:
obtaining an age information and a Coronary Artery Disease (CAD) risk factor information of the user;
wherein determining the MACE occurrence level further comprises:
determining the MACE occurrence level according to the QTc, the age information and the CAD risk factor information.

19. The computer-implemented method of claim 18, wherein determining the MACE occurrence level further comprises:
determining a first score for the age information according to a score table;
determining a second score for the CAD risk factor information according to the score table;
determining a third score for the QTc according to the score table;
determining the MACE occurrence level according to a sum of the first score, the second score and the third score.

20. The computer-implemented method of claim 17, further comprising:
obtaining an age information, a Coronary Artery Disease (CAD) risk factor information of the user, a creatinine information and a troponin information;
wherein determining the MACE occurrence further comprises:
determining the MACE occurrence according to the QTc, the age information, the CAD risk factor information, the creatinine information and the troponin information.

21. The computer-implemented method of claim 20, wherein determining the MACE occurrence level further comprises:
determining a first score for the age information according to a score table;
determining a second score for the CAD risk factor information according to the score table;
determining a third score for the QTc according to the score table;
determining a fourth score for the creatinine information according to the score table;
determining a fifth score for the troponin information according to the score table;
determining the MACE occurrence level according to a sum of the first score, the second score, the third score, the fourth score and the fifth score.
